(19) United States
(12) Patent Application Publication
(10) Pub. No.: US 2013/0164378 A1
(43) Pub. Date: Jun. 27, 2013

(54) Special Prenatal Nutritional Supplement and Design Method Thereof

(71) Applicant: Zhong Ma, Naperville, IL (US)

(72) Inventor: Zhong Ma, Naperville, IL (US)

(21) Appl. No.: 13/820,746

(22) PCT Filed: Sep. 6, 2010
(86) PCT No.: PCT/CN2010/076633

§ 371 Date: Mar. 4, 2013

(51) Int. Cl. A61K 33/00; A61K 35/00

(52) U.S. Cl. CPC A61K 33/00; A61K 35/00
USPC 424/474; 424/600

(57) ABSTRACT

A special prenatal nutritional supplement, characterized as containing active ingredients such as nutrients (including at least one or more vitamins), minerals, or a mixture of pharmaceutical and standard food material. The design method of this special prenatal nutritional supplement is that the type and dosage of the ingredients are defined each day in accordance with the chronological order of the development of the fetus. The product is arranged or displayed like a calendar in a day by day order.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

1. Field of Invention

The present invention belongs to the supplement field, and more particularly to the field of special prenatal supplements and the methods to make the special supplement.

2. Description of Related Arts

There are many varieties of vitamins and minerals to treat specific diseases or to play the important physiological role of nutritional supplements. Vitamins and minerals can a person prevent or recover from diseases, or maintain the body's normal healthy status. It is widely accepted knowledge that insufficiencies of a particular vitamin or mineral can cause certain human diseases. These insufficiencies most greatly affect a human during the stage of embryonic development. The embryonic stage is the most critical period in the formation and development of various tissues and organs of the human body. Congenital defects that occur in this period cannot be improve after birth. For example in human brain tissue cell development, which determines the level of intelligence when the baby is born. A fetus receives its entire nutritional intake from its mother, so the nutritional intake of the mother determines the level of development of the fetus. Nutrition, whether good or bad, plays a critical role in fetal development in pregnant women. If certain nutrients, vitamins, and minerals are lacked, this may lead to disease in the mother and/or the fetus.

Due to the special nutritional needs for pregnant women, pregnant women should take-in more nutrients than non-pregnant woman. However, due to individual living habits and environments, pregnant women's diets are all unique. The nutrient content of food cannot be guaranteed to meet a pregnant woman's special physiological needs, so the minerals, vitamins, and essential nutrients which play a key role in fetal development may be low, and thus it is very common for the pregnant woman to exhibit the symptoms of partial eclipse or anorexia phenomena. Since it is difficult to ensure the proper intake of nutrients, vitamins, and minerals from a usual diet, it is necessary to take nutritional supplements to meet the needs of pregnant woman. At the same time, these nutritional supplements for pregnant woman can help with health and a postpartum recovery.

The continuous supply of traditional nutritional supplements showed imperfections and shortcomings in the nutritional needs of pregnant women. The minerals, vitamins and nutrients provided by traditional nutritional supplements are not targeted or subject oriented. When fetal tissue and organ development are in different stages, the demand for the types and dosages of minerals, vitamins, and nutrients are different. In traditional prenatal supplements, the types and dosage of ingredients (vitamins and minerals) are kept the same.

During embryonic development, some pregnant women sometimes take too much unnecessary minerals, vitamins, and nutrients. This results in the accumulation of some minerals and vitamins, but the long term accumulation of certain vitamins and minerals can cause toxicity in the human body. This is an unnecessary burden during pregnancy, as pregnant woman more sensitivity to this toxicity and this can result in harm to both the pregnant woman and the fetus. Conversely, traditional prenatal supplements can lack the proper supply certain minerals, vitamins and nutrients for a particular stage of embryonic development which will be hazardous for the development of the fetus.

Therefore, a special nutritional supplement for pregnant women must prevent both a lack of nutrient supply and overdose accumulations, as these may have an adverse impact on the healthy development of the fetus.

### SUMMARY OF THE PRESENT INVENTION

The object of the present invention is to avoid the disadvantages of the above conventional prenatal nutritional supplements and to provide a special prenatal nutritional supplement and the method design. This prenatal supplement is completely different from any current prenatal product on the market. The type and dosage of ingredients such as the vitamin, minerals, and other nutrients in this prenatal supplement are defined in accordance with a chronological order of development of a fetus; whereby in this design method, the products are arranged or displayed like a calendar in a day by day order.

Additional advantages and features of the invention will become apparent from the description which follows, and may be realized by means of the instrumentalities and combinations particular point out in the appended claims.

According to the present invention, the foregoing and other objects and advantages are attained by a special prenatal nutritional supplement.

In accordance with another aspect of the invention, the present invention comprises a special prenatal nutritional supplement, characterized as containing active ingredients such as nutrients (including at least one or more vitamins), minerals, or a mixture of pharmaceutical and standard food material. The design method of this special prenatal nutritional supplement is that the type and dosage of the ingredients are defined each day in accordance with the chronological order of the development of the fetus. The product is arranged or displayed like a calendar in a day by day order.

A special prenatal nutritional supplement, characterized as containing active ingredients such as nutrients (including at least one or more vitamins), minerals, or a mixture of pharmaceutical and standard food material. The design method of this special prenatal nutritional supplement is that the type and dosage of the ingredients are defined each week in accordance with chronological order of the development of the fetus. The product is arranged or displayed like a calendar in a day by day order.

A special prenatal nutritional supplement, characterized as containing active ingredients such as nutrients (including at least one or more vitamins), minerals, or a mixture of pharmaceutical and standard food material. The design method of this special prenatal nutritional supplement is that the type and dosage of the ingredients are defined each month in accordance with chronological order of the development of the fetus. The product is arranged or displayed like a calendar in a day by day order.

The dosage form of the prenatal nutritional supplement as mentioned above may be the following: tablets, coated tablets, film-coated tablets, enteric coated tablets, capsules, hard capsules, soft capsules, oral solutions, syrups, chewing agent, fudge, gum, powder, granules, pills, powders, pastes, suspensions, solutions, injections, suppositories, ointments, plasters, creams, spray, drops, patches, sustained release formulations, controlled release formulations, or combinations of these.

The dosage form of the prenatal nutritional supplement as mentioned above may be the following: tablets, coated tablets, film-coated tablets, enteric coated tablets, capsules, hard capsules, soft capsules, oral solutions, syrups, chewing agent, fudge, gum, powder, granules, pills, powders, pastes, suspensions, solutions, injections, suppositories, ointments, plasters, creams, spray, drops, patches, sustained release formulations, controlled release formulations, or combinations of these.

Humans are the most advanced mammals and the normal human embryonic development time frame is substantially constant. According to conventional medical knowledge, a fully developed human embryo requires a total of 266 days. During this dynamic process of 266 days, human embryos strictly obey certain chronological events to complete the development of variety of tissues, organs, and systems. This means that human embryonic development is a dynamic process whereby specific tissues and organ can be only developed during a specific period of time. The mother is the significant source to provide the corresponding nutrients, vitamins, and minerals when embryonic tissue, organ, and system development requirements correspond to the specific relevant period.

For the pregnant women, it is easy to calculate the current gestational stage and determine the number days remaining in the pregnancy after conception. The mother can then start to take the new prenatal nutritional supplement corresponding to the gestational stage.

The benefit of the present invention is that the type and dosage of the vitamins, minerals, and nutrients in this prenatal nutritional supplement match the demand for the vitamins, minerals and nutrients for particular fetal tissues, organs, and body systems development at a certain time frame.

The present invention solves the disadvantage of the traditional prenatal nutritional supplements of satisfying the type and dosage demands for vitamins, minerals, and nutrients without causing the accumulation of certain minerals and vitamins, and thus no harm will come to the fetus or mother. This product is also arranged like a calendar which prompts the advantage presenting when the proper dosage must be taken. This makes sure that pregnant women can get the continued supply of the needed vitamins and other nutrients in the fetal development process, and thus ensure the healthy development of the fetus.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

Example 1 is a design method of this prenatal nutritional supplement when the type and dosage of ingredients are defined each day in accordance with the chronological order of fetus development.

Example 2 is a design method of this prenatal nutritional supplement when the type and dosage of the ingredients are defined each month in accordance with the chronological order of fetus development.

Example 3 is a design method of this prenatal nutritional supplement when the types and dosages of the ingredients are defined each week in accordance with the chronological order of fetus development.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

### Example 1

The design method of this prenatal nutritional supplement when the type and dosage of ingredients are defined each day in accordance with the chronological order of fetus development.

In this design method the pregnant women's special nutritional supplement is taken in the form of a capsule, wherein all capsules are sealed in blister pads and arranged to form 40 lines, each of which represents the demand of one week. These lines would be numbered 1 through 40 to correspond to the appropriate week of fetal development. Each of these 40 lines would have 7 dosages associated with each week to represent each of the days per week. Each of these capsules would be labeled 1 to 280 to represent each day of the 40 weeks. Since the supplement products are to be arranged like a calendar in a day by day order, the types and dosages of the active ingredients for each day are defined by each day in the process of fetal development. Therefore, the type and dosage for each day are different.

Pregnant women can calculate their current stage of pregnancy (the days after conception). For example, when a pregnant woman calculates that their current stage of pregnancy has been 30 days, she then can find the capsule corresponding to the number 30, and then start taking the capsules from this point every day until the 280th capsule is finished.

### Example 2

The design method of this prenatal nutritional supplement when the type and dosage of the ingredients are defined each month in accordance with the chronological order of fetus development.

In this design method, the pregnant women's special nutritional supplement are taken in the form of a capsule, wherein all the capsules are sealed in blister pads and arranged to form 40 lines, each of which represents the demand of one week. All these lines would represent 40 weeks dosage, where every 4 weeks is assigned as one month and each of these months would be assigned a number corresponding to “number of the month”.

The lines are numbered from 1 to 10 corresponding to each of the 10 months of the dosage. Each of the lines would have 7 dosages corresponding to each of the days per week. Each capsule in a blister pad would be labeled with a number from 1 to 280 corresponding to each day of the 10 months. Since the supplement products are to be arranged like a calendar in a day by day order, the types and dosages of the active ingredients for each day are defined by each month in the process of fetal development; therefore, for capsules sealed in every 4 lines, the type and dosage of the active ingredients each of the capsules are the same. That means that each month (4 weeks), the type and dosage of the active ingredients in each of the capsules are the same. However, the types and dosages of the active nutrients in the capsules for each month are different.

Pregnant women can calculate their current stage of pregnancy (days after conception). For example, when a pregnant woman calculates that their current stage of pregnancy stage is 30, she then can find the capsule corresponding to 30, and then start taking the capsules from this point every day until the 280th capsule is finished.

### Example 3

The design method of this prenatal nutritional supplement when the types and dosages of the ingredients are defined each week in accordance with the chronological order of fetus development.

In this design method, the pregnant women's special nutritional supplement are taken in the form of a capsule, wherein all the capsules are sealed in blister pads and arranged to form 40 lines. Each of these 40 lines represents the dosage demand of one week. The lines are numbered from 1 to 40 and each line would have 7 dosages which would correspond to the 7 days per week. Each capsule in a blister pad will be labeled from to 280 to represent the demand for the corresponding capsules from day 1 to day 280. Since the supplement products are to be arranged like a calendar in a day by day order, the type and dosage of the active ingredients for each day are defined by each week in the process of fetal development. Therefore, the type and dosage of the active ingredients for capsules sealed in the same line are the same. That means that the type and dosage of the active ingredients for capsules sealed in the same line are the same. But for different weeks, the type and dosage of the active ingredients of the capsules are different.

Pregnant woman can calculate their current stage of pregnancy (days after conception). For example, when a pregnant woman calculates that their current stage of pregnancy is 30 days, then she can find the capsule corresponding to 30 and from this point start taking a capsule every day until the 280th capsule is finished.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A special prenatal nutritional supplement, characterized as having the active ingredients of nutrients (including at least one or more vitamins), minerals, or a mixture of pharmaceutical and standard food materials.

2. The design method of this special prenatal nutritional supplement which is described in claim 1, characterized by the type and dosage of ingredients defined each day in accordance with the chronological development of the fetus, wherein the product is arranged or displayed like a calendar in a day by day order.

3. The design method of this special prenatal nutritional supplement which is described in claim 1, characterized by the type and dosage of ingredients defined each week in accordance with the chronological development of the fetus, wherein the product is arranged or displayed like a calendar in a day by day order.

4. The design method of this special prenatal nutritional supplement which is described in claim 1, characterized by the type and dosage of ingredients defined each month in accordance with the chronological development of the fetus, wherein the product is arranged or displayed like a calendar in a day by day order.

5. According to above claims 2 thru 4, any one of the design methods of this special prenatal nutritional supplement, characterized by the dosage forms such as tablets, coated tablets, film-coated tablets, enteric coated tablets, capsules, hard capsules, soft capsules, oral solutions, syrups, chewing agent, fudge, gum, powder, granules, pills, powders, pastes, suspensions, solutions, injections, suppositories, ointments, plasters, creams, spray, drops, patches, sustained release formulations, controlled release formulations, and combinations of these.
